(19) United States
(12) Patent Application Publication
(10) Pub. No.: US 2026/0315097 A1
(43) Pub. Date: Oct. 15, 2026

(54) SURGICAL INSTRUMENTS AND SYSTEMS INCLUDING JAW ACTUATION MECHANISMS

(71) Applicant: Covidien LP, Mansfield, MA (US)

(72) Inventors: Matthew S. Cowley, Frederick, CO (US); Michael B. Lyons, Boulder, CO (US)

(21) Appl. No.: 19/689,076

(22) Filed: May 27, 2026

Related U.S. Application Data
(63) Continuation of application No. PCT/IB2024/061853, filed on Nov. 26, 2024.
(60) Provisional application No. 63/605,236, filed on Dec. 1, 2023.

(51) Int. Cl. A61B 17/32; A61B 17/00

(52) U.S. Cl. CPC A61B 17/320068; A61B 2017/00477; A61B 2017/320075

(57) ABSTRACT

An ultrasonic surgical instrument includes a curved ultrasonic blade, a jaw member, and a jaw actuation mechanism. The jaw member is movable between open and closed positions for clamping tissue against a top side of the blade and includes first and second proximal flags disposed on respective concave and convex sides of the blade. The first flag defines a portion that extends beyond the second flag. The jaw actuation mechanism includes a first arm pivotably connected to the first and second flags on the top side of the blade and a second arm operably coupled to the portion of the first flag on the concave side of the blade. Movement of the first and/or second arm relative to the other moves the jaw member between the open and closed positions.

### CROSS-REFERENCE TO RELATED APPLICATIONS

This application is a continuation of International Patent Application No. PCT/IB2024/061853, filed Nov. 26, 2024, which claims the benefit of U.S. Provisional Patent Application Ser. No. 63/605,236, filed Dec. 1, 2023, the entire contents of each of which applications are incorporated herein by reference. To the extent appropriate, a claim of priority is made to each of the above disclosure applications.

### FIELD

The present disclosure relates to surgical instruments and systems and, more particularly, to surgical instruments and systems including jaw actuation mechanisms to facilitate clamping and treating tissue.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical instrument may include, for example, an ultrasonic blade and a jaw member to enable clamping of tissue against the blade. Ultrasonic energy transmitted to the blade causes the blade to vibrate at very high (e.g., ultrasonic) frequencies, which allows for heating tissue clamped against or otherwise in contact with the blade to treat the tissue.

Other energy modalities such as, for example, Radio Frequency (RF) energy, thermal energy, microwave energy, and light energy, may additionally or alternatively be utilized to treat tissue clamped between opposing structures.

### SUMMARY

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations and tolerances, up to and including plus or minus 10 percent. Further, any or all of the aspects described herein, to the extent consistent, may be used in conjunction with any or all of the other aspects described herein.

Provided in accordance with aspects of the present disclosure is an ultrasonic surgical instrument including a curved ultrasonic blade, a jaw member, and a jaw actuation mechanism. The curved ultrasonic blade includes a top side, a bottom side, a convex side, and a concave side. The jaw member is movable between an open position and a closed position for clamping tissue against the top side of the curved ultrasonic blade. The jaw member includes a first proximal flag disposed on the concave side of the curved ultrasonic blade and a second proximal flag disposed on the convex side of the curved ultrasonic blade. The first proximal flag defines a height greater than a height of the second proximal flag such that a portion of the first proximal flag extends beyond the second proximal flag. The jaw actuation mechanism includes a first arm pivotably connected to the first proximal flag and the second proximal flag on the top side of the curved ultrasonic blade and a second arm operably coupled to the portion of the first proximal flag on the concave side of the curved ultrasonic blade. Movement of at least one of the first arm or the second arm relative to another of the first arm or the second arm moves the jaw member between the open position and the closed position.

In an aspect of the present disclosure, the second arm includes a first upright and a second upright defining a channel therebetween. In such aspects, the portion of the first proximal flag is received within the channel.

In another aspect of the present disclosure, at least one of the first upright or the second upright defines a slot, and the portion of the first proximal flag includes a cam received within the slot.

In yet another aspect of the present disclosure, the first upright defines a spring finger configured to maintain the portion of the first proximal flag in alignment within the channel.

In still another aspect of the present disclosure, the ultrasonic surgical instrument further includes a first shaft and a second shaft. The first arm is fixed relative to and extends distally from the first shaft and the second arm is fixed relative to and extends distally from the second shaft. One of the first shaft or the second shaft is movable relative to another of the first shaft or the second shaft to thereby move the at least one of the first arm or the second arm relative to the other of the first arm or the second arm.

In still yet another aspect of the present disclosure, the first arm includes a portion that conforms to a portion of an outer periphery of the first shaft and/or the second arm includes a portion that conforms to a portion of an outer periphery of the second shaft.

In another aspect of the present disclosure, the first proximal flag defines a substantially planar configuration, and the second proximal flag includes a curved portion.

In still another aspect of the present disclosure, the first arm includes a curved portion, and the curved portion of the second proximal flag is complementary to the curved portion of the first arm such that the curved portions of the first arm and the second proximal flag cooperate to guide the jaw member during movement of the jaw member between the open position and the closed position.

In yet another aspect of the present disclosure, the second proximal flag further includes a substantially planar portion extending from the curved portion and the first arm includes a substantially planar portion extending from the curved portion. In such aspects, the substantially planar portions of the first arm and the second proximal flag cooperate to guide the jaw member during movement of the jaw member between the open position and the closed position.

In still yet another aspect of the present disclosure, a pivot pivotably connects the first arm to the first proximal flag and the curved portion of the second proximal flag.

Another ultrasonic surgical instrument provided in accordance with aspects of the present disclosure includes a shaft assembly, an ultrasonic blade, and a jaw member. The shaft assembly includes a first shaft and a second shaft. At least one of the first shaft or the second shaft is movable relative to another of the first shaft or the second shaft. The ultrasonic blade extends distally from the shaft assembly and includes a top side, a bottom side, a first lateral side, and a second lateral side. The jaw member extends distally from the shaft assembly, is pivotably mounted relative to the second shaft about a pivot, and is movable about the pivot between an open position and a closed position for clamping tissue against the top side of the ultrasonic blade. The jaw member is operably coupled to the first shaft only on the first lateral side of the ultrasonic blade and is operably coupled to the first shaft such that the movement of the at least one of the first shaft or the second shaft relative to the other of the first shaft or the second shaft moves the jaw member about the pivot between the open position and the closed position.

In an aspect of the present disclosure, an actuation arm operably couples the jaw member with the first shaft only on the first lateral side of the ultrasonic blade. The actuation arm includes a first upright and a second upright defining a channel therebetween. The jaw member includes a flag received within the channel.

In another aspect of the present disclosure, at least one of the first upright or the second upright defines a slot, and the flag includes a cam received within the slot.

In another aspect of the present disclosure, the first upright defines a spring finger configured to maintain the flag in alignment within the channel.

In still another aspect of the present disclosure, the actuation arm is fixed relative to and extends distally from the first shaft. Additionally or alternatively, a portion of the actuation arm defines an extension of an outer periphery of a portion of the first shaft.

In yet another aspect of the present disclosure, a pivot arm pivotably couples the jaw member with the second shaft. The pivot arm is disposed on the top side of the ultrasonic blade.

In still yet another aspect of the present disclosure, the pivot arm and the jaw member define complementary at least partially curved guide surfaces only on the second lateral side of the ultrasonic blade.

In another aspect of the present disclosure, a portion of the pivot arm defines an extension of an outer periphery of a portion of the second shaft.

In another aspect of the present disclosure, the ultrasonic blade is curved such that the first lateral side is concave and the second lateral side is convex.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument and a surgical generator;

FIG. 2 is perspective view of another surgical system provided in accordance with the present disclosure including a surgical instrument incorporating one or more surgical generators and one or more power sources;

FIG. 3 is a schematic illustration of a robotic surgical system provided in accordance with the present disclosure;

FIG. 4 is a side view of an end effector assembly provided in accordance with the present disclosure and configured for use with the surgical system of FIG. 1, the surgical system of FIG. 2, the robotic surgical system of FIG. 3, or any other suitable surgical system;

FIG. 5A is a top view of the end effector assembly of FIG. 4;

FIG. 5B is a bottom, mirror image view of the end effector assembly of FIG. 4;

FIGS. 6A and 6B are front and front transverse cross-sectional views, respectively, of the end effector assembly of FIG. 4;

FIG. 7A is a side, perspective view of the end effector assembly of FIG. 4 with an ultrasonic blade of the end effector assembly removed;

FIG. 7B is a side, perspective view of the end effector assembly of FIG. 4 with the ultrasonic blade and a jaw member of the end effector assembly removed;

FIG. 8A is a side, perspective view of another end effector assembly provided in accordance with the present disclosure and configured for use with the surgical system of FIG. 1, the surgical system of FIG. 2, the robotic surgical system of FIG. 3, or any other suitable surgical system, with an ultrasonic blade of the end effector assembly removed; and

FIG. 8B is a front view of the end effector assembly of FIG. 8A with the ultrasonic blade and a jaw member of the end effector assembly removed.

### DETAILED DESCRIPTION

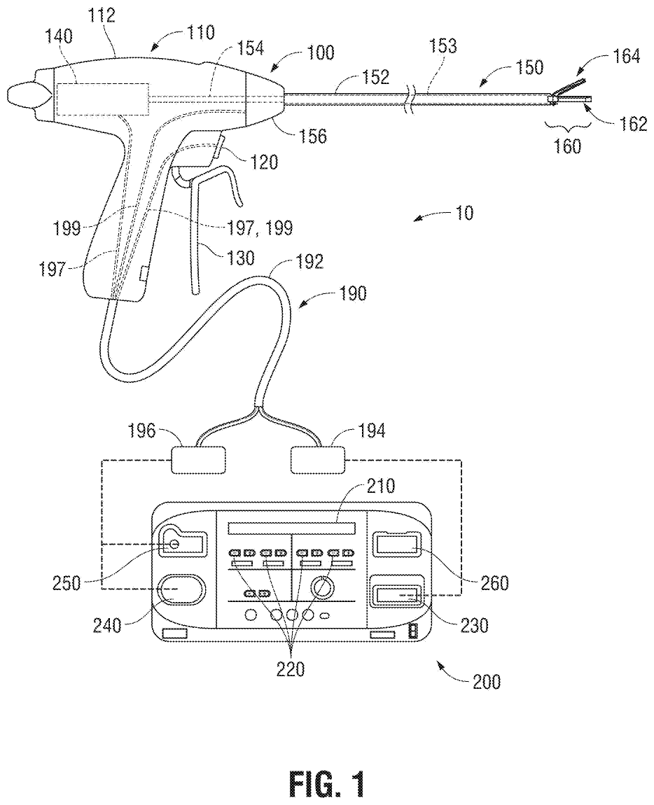

Referring to FIG. 1, a surgical system provided in accordance with aspects of the present disclosure is shown generally identified by reference numeral 10 including a surgical instrument 100 and a surgical generator 200. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality user interface features 220, e.g., buttons, touch screens, switches, etc., an ultrasonic energy plug port 230, and an electrosurgical energy plug port 240 (which may be a monopolar Radio Frequency (RF) energy plug port for supplying monopolar energy, a bipolar RF energy plug port for supplying bipolar energy, or any other suitable electrical energy plug port). Surgical generator 200 may further include additional ports 250, 260 to provide additional functionality, e.g., a monopolar plug port, a bipolar plug port, an electrical plug port to power a thermal heating element, a microwave plug port for supplying microwave energy, a vacuum plug port enabling suction, a fluid plug port enabling irrigation, additional power ports for supplying AC and/or DC electrical power, etc. As an alternative to plural dedicated ports 230-260, one or more common ports (not shown) may be configured to act as any two or more of ports 230-260.

Surgical instrument 100 is configured to supply ultrasonic energy to ultrasonic blade 162 of end effector assembly 160, e.g., for treating tissue in contact with ultrasonic blade 162. In aspects, surgical instrument 100 is further configured to supply RF to end effector assembly 160 for treating tissue in contact with ultrasonic blade 162 and/or jaw member 164, together with the application of ultrasonic energy or separately therefrom. To enable RF tissue treatment, surgical generator 200 is configured to provide RF energy for output through the electrosurgical energy plug port 240 to energize ultrasonic blade 162 and/or jaw member 164 and conduct of RF energy (in a monopolar or bipolar configuration) through tissue in contact with ultrasonic blade 162 and/or jaw member 164 to heat and thereby treat the tissue. To enable ultrasonic tissue treatment, surgical generator 200 is configured to provide ultrasonic drive signals for output through ultrasonic plug port 230 to surgical instrument 100 to drive ultrasonic transducer 140 to produce ultrasonic energy for transmission along ultrasonic waveguide 154 to ultrasonic blade 162 of end effector assembly 160 to heat and thereby treat the tissue in contact with ultrasonic blade 162. In aspects, RF energy capability is omitted or replaced with any other suitable additional energy modality such as, for example, microwave energy, light energy, thermal energy, etc.

Continuing with reference to FIG. 1, handle assembly 110 includes a housing 112, an activation button 120, and a trigger 130. Housing 112 is configured to support ultrasonic transducer 140. Ultrasonic transducer 140 may be permanently engaged within housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along ultrasonic waveguide 154 of elongated assembly 150 to ultrasonic blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF state to different ON states corresponding to different activation settings, e.g., a first ON state corresponding to a first activation setting (such as a LOW power and/or tissue sealing setting) and a second ON state corresponding to a second activation setting (such as a HIGH power and/or tissue transection setting). In still other configurations, separate activation buttons may be provided, e.g., a first actuation button for activating a first activation setting and a second activation button for activating a second activation setting. Additional activation buttons, sliders, wheels, etc. are also contemplated to enable control of various different activation settings from housing 112.

Elongated assembly 150 of surgical instrument 100 includes an outer shaft 152, an inner shaft 153 disposed within outer shaft 152, ultrasonic waveguide 154 extending through inner shaft 153, a drive assembly (not shown), a rotation knob 156, and end effector assembly 160 including ultrasonic blade 162 and jaw member 164. Rotation knob 156 is rotatable in either direction to rotate elongated assembly 150 in either direction relative to handle assembly 110. The drive assembly operably couples a proximal portion of outer shaft 152 to trigger 130 of handle assembly 110. A distal portion of outer shaft 152 is operably coupled to jaw member 164 and a distal end of inner shaft 153 pivotably supports jaw member 164. As such, trigger 130 is selectively actuatable, e.g., from an un-actuated position towards an actuated position, to thereby actuate the drive assembly to move outer shaft 152 about inner shaft 153 to pivot jaw member 164 relative to ultrasonic blade 162 of end effector assembly 160 from an open position towards a closed position for clamping tissue between jaw member 164 and ultrasonic blade 162. In aspects, the configuration of outer and inner shafts 152, 153 may be reversed, e.g., wherein outer shaft 152 is the support shaft and inner shaft 153 is the drive shaft.

Referring still to FIG. 1, the drive assembly may be tuned to provide a jaw clamping force, or jaw clamping force within a jaw clamping force range, to tissue clamped between jaw member 164 and ultrasonic blade 162, such as described in U.S. Patent Application Pub. No. 2022/0117622, the entire contents of which are hereby incorporated herein by reference. Alternatively, the drive assembly may include a force limiting feature, e.g., a spring, whereby the clamping force applied to tissue clamped between jaw member 164 and ultrasonic blade 162 is limited to a particular jaw clamping force or a jaw clamping force within a jaw clamping force range, such as described in U.S. Pat. No. 10,368,898, the entire contents of which are hereby incorporated herein by reference.

Continuing with reference to FIG. 1, ultrasonic waveguide 154, as noted above, extends from handle assembly 110 through inner shaft 153. Ultrasonic waveguide 154 includes ultrasonic blade 162 disposed at a distal end thereof. Ultrasonic blade 162 may be integrally formed with waveguide 154, separately formed and subsequently attached (permanently or removably) to ultrasonic waveguide 154, or otherwise operably coupled with ultrasonic waveguide 154. Ultrasonic waveguide 154 and/or ultrasonic blade 162 may be formed from titanium, a titanium alloy, or other suitable electrically conductive material(s), although non-conductive materials are also contemplated. Ultrasonic waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along ultrasonic waveguide 154 to ultrasonic blade 162 for treating tissue clamped between ultrasonic blade 162 and jaw member 164 or positioned adjacent to ultrasonic blade 162.

Cable assembly 190 of surgical instrument 100 includes a cable 192, an ultrasonic plug 194, and, in aspects where RF energy (or other additional energy modality) is provided, an electrosurgical plug 196. Ultrasonic plug 194 is configured for connection with ultrasonic plug port 230 of surgical generator 200 while electrosurgical plug 196 is configured for connection with electrosurgical plug port 240 of surgical generator 200. In configurations where generator 200 includes a common port, cable assembly 190 may include a common plug (not shown) configured to act as both the ultrasonic plug 194 and the electrosurgical plug 196.

Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of ultrasonic energy. In addition, plural second electrical lead wires 199 are electrically coupled to electrosurgical plug 196 and extend through cable 192 into handle assembly 110. One or more second electrical lead wires 199 electrically connects to ultrasonic waveguide 154 within handle assembly 110 and/or one or more second electrical lead wires 199 extends through handle assembly 110 and elongated assembly 150 to electrically connect to jaw member 164. In bipolar configurations, for example, isolated second electrical lead wires 199 may connect to isolated portions of jaw member 164 to enable energization of the isolated portions to different potentials for the conduction of RF energy therebetween and through tissue, or isolated second electrical lead wires 199 may connect to ultrasonic waveguide 154 and jaw member 164 to enable energization of the isolated portions to different potentials for the conduction of RF energy therebetween and through tissue. In monopolar configurations, ultrasonic waveguide 154 and/or jaw member 164 may be energized via one or more second electrical lead wires 199 and energy may be returned locally or remotely. In any of the above configurations, one or more other second electrical lead wires 199 may be electrically coupled to activation button 120 to enable the selective supply of RF energy from surgical generator 200 to ultrasonic waveguide 154 and/or jaw member 164 upon activation.

Continuing with reference to FIG. 1, with respect to activation of RF energy, similarly as detailed above with respect to ultrasonic activation, activation button 120 (or an additional activation button) is coupled to or between ultrasonic waveguide 154 and/or jaw member 164 and surgical generator 200, e.g., via one or more of second electrical lead wires 199, to enable the supply of RF energy in response to depression of activation button 120. One or more of the ON states of activation button 120 may correspond to one or more activation settings of RF energy, e.g., a first ON state corresponding to a first activation setting (such as a LOW power mode) and a second ON state corresponding to a second activation setting (such as a HIGH power mode). Multiple actuation buttons for activating the different activation settings and/or additional activation buttons, sliders, wheels, etc. are also contemplated to enable control of various different activation settings from housing 112.

With respect to both ultrasonic activation and activation of RF energy, activation button 120 and/or additional activation buttons, sliders, wheels, etc. may be provided for enabling selective activation of: only ultrasonic energy (in one or more different modes thereof); only RF energy (in one or more different modes thereof); and/or a combination of ultrasonic energy and RF energy (in one or more different modes such as, for example, simultaneous activation, alternating activation, consecutive activation, overlapping activation, and/or according to any other suitable activation protocol or algorithm).

Generator 200 may further be configured to provide energy control and/or sensing functionality based upon feedback from the electrosurgical energy. More specifically, by sensing the current, voltage, power, resistance, and/or other electrical properties associated with the RF energy circuit(s), generator 200 is capable of: controlling the application of electrosurgical and/or ultrasonic energy in accordance with a feedback algorithm; determining an impedance of tissue; determining a location, size, and/or type of tissue clamped between jaw member 164 and/or ultrasonic blade 162; and/or determining a progress of tissue treatment (e.g., tissue sealing in progress, tissue is fully sealed, tissue is sealed and transected, etc.).

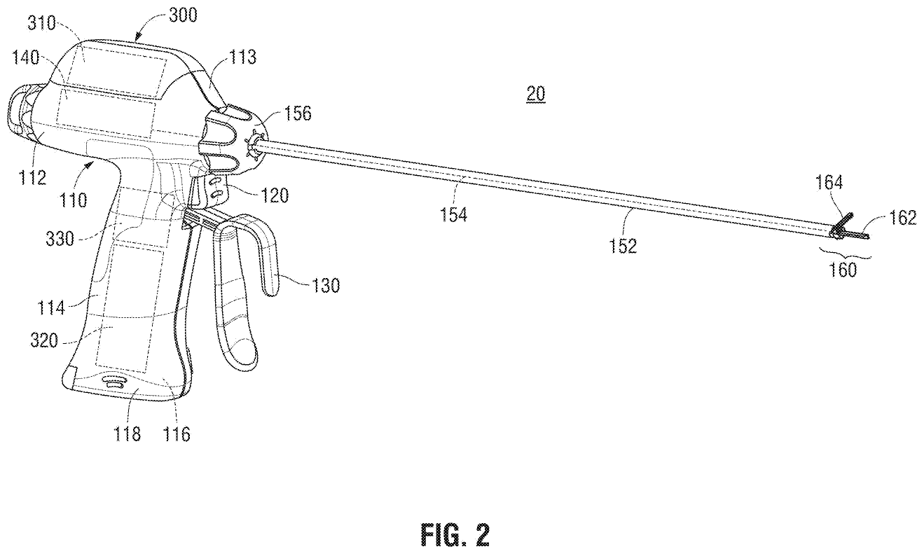

As an alternative to a remote generator 200, surgical system 10 may be at least partially cordless in that it incorporates an ultrasonic generator, an electrosurgical generator (or suitable electrosurgical circuitry), and/or a power source, e.g., a battery, thereon or therein. In this manner, the connections from surgical instrument 100 to external devices, e.g., generator(s) and/or power source(s), are reduced or eliminated. More specifically, with reference to FIG. 2, another surgical system in accordance with the present disclosure is shown illustrated as a surgical instrument 20 supporting an ultrasonic generator 310, a power source (e.g., battery assembly 320), and an electrosurgical generator 330 thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 320 and electrosurgical generator 330 and a door 118 configured to enclose compartment 116. An electrical connection assembly (not shown) is disposed within housing 112 and serves to electrically couple activation button 120, ultrasonic generator 310 of TAG 300, and battery assembly 320 with one another when TAG 300 is supported on or in body portion 113 of housing 112 and battery assembly 320 is disposed within compartment 116 of fixed handle portion 114 of housing 112, thus enabling activation of surgical instrument 20 in an ultrasonic mode in response to appropriate actuation of activation button 120. Further, the electrical connection assembly or a different electrical connection assembly disposed within housing 112 serves to electrically couple activation button 120, electrosurgical generator 330, battery assembly 320, and end effector assembly 160 with one another when electrosurgical generator 330 and battery assembly 320 are disposed within compartment 116 of fixed handle portion 114 of housing 112, thus enabling activation of surgical instrument 20 to supply electrosurgical energy, e.g., RF energy, to end effector assembly 160 in response to appropriate actuation of activation button 120. In aspects, electrosurgical generator 330 is omitted and some or all of the above functionality is provided by battery assembly 320.

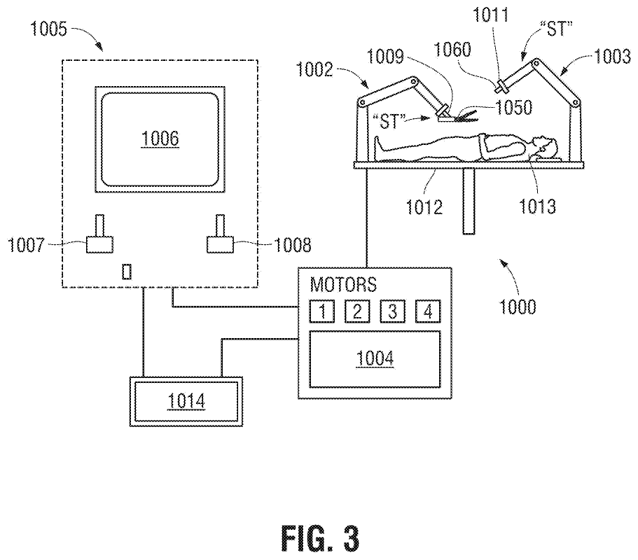

Referring to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display video and/or three dimensional images; and manual input devices 1007, 1008, by means of which a clinician (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive or other suitable manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching drive unit 1009, 1011, to which may be attached, for example, a surgical tool “ST” supporting an end effector assembly 1050, 1060. One of the surgical tools “ST” may be surgical instrument 100 (FIG. 1), surgical instrument 20 (FIG. 2), or any other suitable surgical instrument, wherein manual actuation features, e.g., actuation button 120 (FIG. 1), trigger 130 (FIG. 1), etc., are replaced with robotic input couplers to direct the attaching drive unit 1009, 1011 to activate and/or manipulate the end effector assembly 1050, 1060 in a desired manner. Robotic surgical system 1000 may include or be configured to connect to an ultrasonic generator, an electrosurgical generator, and/or a power source (whether separate components or housed together in a single console). The other surgical tool “ST” may include any other suitable surgical instrument, e.g., an endoscopic camera, surgical grasper, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

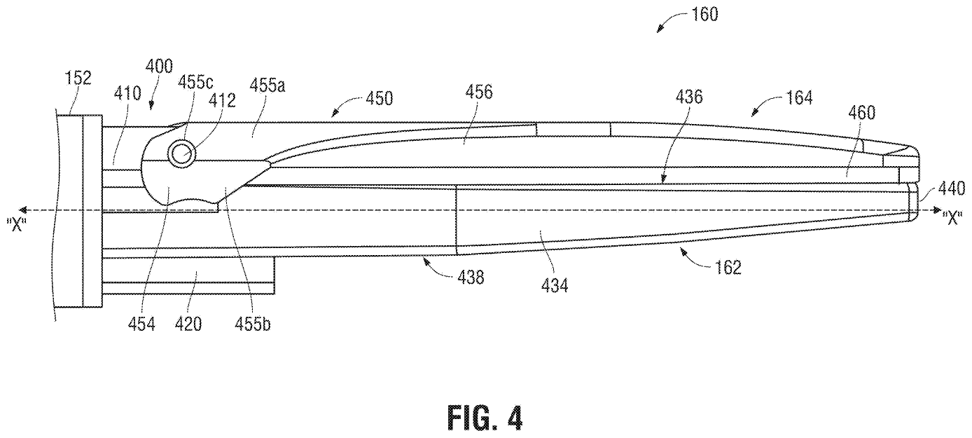

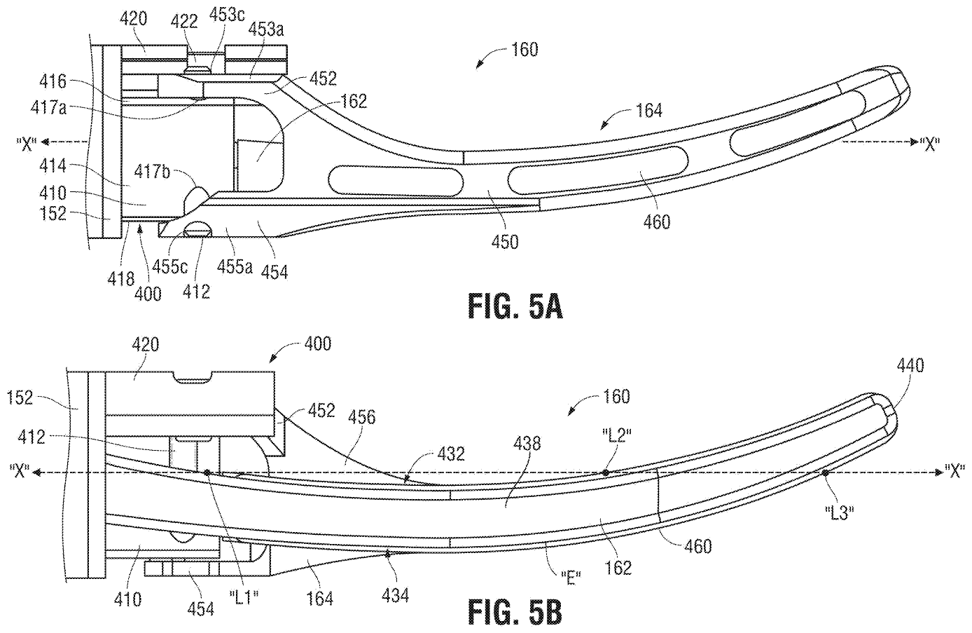

Turning to FIGS. 4-5B, end effector assembly 160 of surgical instrument 100 of surgical system 10 (FIG. 1) is detailed, although the aspects and features of end effector assembly 160 may similarly apply to and be utilized with surgical instrument 20 (FIG. 2), robotic surgical system 1000 (FIG. 3) and/or any other suitable surgical instrument or system.

End effector assembly 160 is operably disposed at distal end portions of outer and inner shafts 152, 153 and, as noted above, includes ultrasonic blade 162 and jaw member 164. End effector assembly 160 further includes a jaw actuation mechanism 400 to enable the selective movement of jaw member 164 relative to blade 162 between the open position and the closed position for clamping tissue therebetween, e.g., in response to actuation of trigger 130 (FIG. 1). Jaw actuation mechanism 400, as described in greater detail below, includes a first arm 410 extending from inner shaft 153 to pivotably mount jaw member 164 at the distal end portion of inner shaft 153 and a second arm 420 extending from outer shaft 152 to operably couple jaw member 164 to the distal end portion of outer shaft 152 such that movement (e.g., slidable motion) of outer shaft 152 about and relative to inner shaft 153 pivots jaw member 164 relative to ultrasonic blade 162 between the open position and the closed position. Further, as also described in greater detail below, jaw actuation mechanism 400 defines an asymmetric configuration with respect to a vertical plane defined through longitudinal axis “X” of outer shaft 152 (and, in aspects, of inner shaft 153 and/or ultrasonic waveguide 154 (FIG. 1), in aspects where inner shaft 153 and/or ultrasonic waveguide 154 (FIG. 1) are coaxially disposed within outer shaft 152) to accommodate the curvature of ultrasonic blade 162.

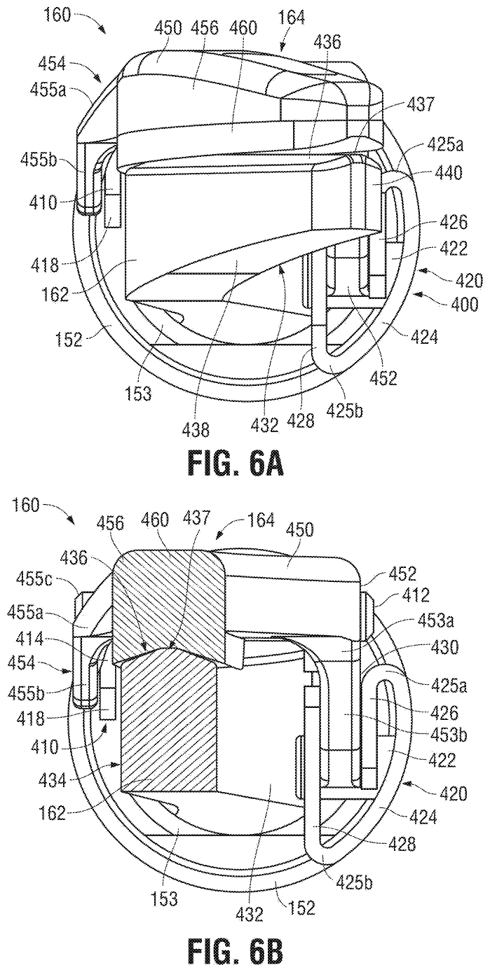

With additional reference to FIGS. 6A and 6B, ultrasonic blade 162 defines first and second side surfaces 432, 434, a top surface 436, and a bottom surface 438. Ultrasonic blade 162 is curved such that first side surface 432 is concave and such that second side surface 434 is convex. More specifically, the curvature of ultrasonic blade 162, in a proximal-to-distal direction, is initially curved laterally outwardly in a first direction relative to longitudinal axis “X” and subsequently curved laterally outwardly in a second, opposite direction relative to longitudinal axis “X” such that a laterally outward-most extent “E” of convex second side surface 434 relative to longitudinal axis “X” is spaced apart from each of the proximal and distal ends of ultrasonic blade 162 and such that the laterally outward-most extent “E” of convex second side surface 434 is disposed on a first side of longitudinal axis “X” while a distal tip 440 of ultrasonic blade 162 is disposed on a second, opposite side of longitudinal axis “X.”

Further, in aspects, the curvature of ultrasonic blade 162 is such that concave first side surface 432 of ultrasonic blade 162 intersects longitudinal axis “X” at two longitudinally spaced locations “L1” and “L2.” Convex second side surface 434 intersects longitudinal axis “X” at a third location “L3” distally spaced from each of the first and second locations “L1” and “L2,” respectively. In aspects, as shown, convex second side surface 434 only intersects longitudinal axis “X” at one location (e.g., third location “L3”); in other aspects, convex second side surface 434 intersects longitudinal axis “X” at a fourth location proximally spaced from each of the first and second locations “L1” and “L2,” respectively.

The curvature of ultrasonic blade 162 begins at a longitudinal location that is proximal of or longitudinally overlaps first arm 410 of jaw actuation mechanism 400. In aspects, the curvature of ultrasonic blade 162 begins within inner shaft 153 (proximally of first arm 410). As a result of the longitudinal location of the initiation of curvature of ultrasonic blade 162 and/or the above-detailed configuration of the curvature of ultrasonic blade 162, clearance between ultrasonic blade 162 and jaw actuation mechanism 400 is more limited on the convex second side surface 434 of ultrasonic blade 162 (e.g., on the first side of longitudinal axis “X”) as compared to the concave first side surface 432 of ultrasonic blade 162 (e.g., on the second side of longitudinal axis “X”). The asymmetric configuration of jaw actuation mechanism 400, as detailed below, accommodates this clearance differential without compromising the functionality of jaw actuation mechanism 400.

Continuing with reference to FIGS. 4-6B, ultrasonic blade 162 may define a polygonal, rounded polygonal, or any other suitable cross-sectional configuration, including various different cross-sectional configurations along its length. Ultrasonic waveguide 154 or at least the portion of ultrasonic waveguide 154 proximally adjacent ultrasonic blade 162, may define a cylindrical shaped configuration. Plural tapered surfaces (not shown) may interconnect the cylindrically shaped ultrasonic waveguide 154 with the polygonal (rounded edge polygonal, or other suitable shape) configuration of ultrasonic blade 162 to define smooth transitions between the body of ultrasonic waveguide 154 and ultrasonic blade 162.

Ultrasonic blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

In addition to the above-detailed curvature of ultrasonic blade 162, ultrasonic blade 162 may be formed to include any other suitable features, e.g., a tapered configuration (laterally and/or vertically), various different cross-sectional configurations along its length, cut outs, indents, edges, protrusions, straight surfaces, curved surfaces, angled surfaces, wide edges, narrow edges, and/or other features. More specifically, in aspects, top surface 436 of ultrasonic blade 162 includes a pair of longitudinally extending surface portions that converge to an apex 437 extending along a length of blade 162. Apex 437 may define a curved surface or a flat surface and is configured to facilitate tensioning tissue for treating, e.g., sealing and transecting, tissue without transecting or prematurely transecting tissue when sealing tissue is desired. Bottom surface 438 may define a flat, substantially planar configuration, although other configurations are also contemplated such as, for example, a configuration that mirrors the above-detailed configuration of top surface 436.

Referring still to FIGS. 4-6B, jaw member 164 includes a structural frame 450 and a jaw liner 460 secured to structural frame 450. Structural frame 450 is configured to provide structural rigidity and support to jaw member 164 and to operably couple jaw member 164 with jaw actuation mechanism 400 to enable selective movement of jaw member 164 relative to ultrasonic blade 162 between the open and closed positions for clamping tissue between jaw liner 460 and ultrasonic blade 162, e.g., in response to sliding movement of outer shaft 152 about and relative to inner shaft 153.

Structural frame 450 of jaw member 164, more specifically, includes first and second proximal flags 452, 454 and a distal body 456 extending distally from proximal flags 452, 454. Structural frame 450 may be formed from a metal, e.g., stainless steel, or other suitable material capable of providing sufficient structural support and rigidity. Structural frame 450 may be monolithically formed from a single piece of material such as, for example, via metal injection molding (MIM), machining, stamping, combinations thereof, etc.

First and second proximal flags 452, 454, respectively, of structural frame 450 are laterally spaced apart from one another and configured to permit passage of ultrasonic blade 162 therebetween. First and second proximal flags 452, 454, more specifically, include upper portions 453a, 455a connected to distal body 456 and lower portions 453b, 455b depending from upper portions 453a, 455a. Upper and lower portions 453a, 453b of first proximal flag 452 cooperate to define a substantially planar configuration of first proximal flag 452 that is oriented substantially vertically. Upper portion 455a of second proximal flag 454 is curved outwardly and downwardly from distal body 456 to lower portion 455b of second proximal flag 454, which defines a substantially planar, substantially vertically oriented configuration.

Upper portions 453a, 455a of first and second proximal flags 452, 454, respectively, define aligned pivot apertures 453c, 455c configured to receive pivot 412 to pivotably mount jaw member 164 to first arm 410 of jaw actuation mechanism 400 and, thus, to the distal end portion of inner shaft 153. Pivot 412 may be fixed within pivot apertures 453c, 455c (e.g., via welding, mechanical capping, etc.) and rotatable relative to first arm 410, may be fixed relative to first arm 410 (e.g., via welding) and rotatable within pivot apertures 453c, 455c, or may be rotatable both relative to first arm 410 and within pivot apertures 453c, 455c. Pivot 412 may include any suitable pivoting structure(s) such as, for example, a continuous pivot pin, a split pivot pin, separate pivot pins, pivot bosses, etc. The location of pivot 412 is spaced apart from and above the longitudinal axis “X,” as shown in FIG. 4.

As noted above, lower portions 453b, 455b of first and second proximal flags 452, 454, respectively, extend from respective upper portions 453a, 455a of first and second proximal flags 452, 454. More specifically, lower portion 453b of first proximal flag 452 extends from upper portion 453a at a location offset above the longitudinal axis “X,” crosses a horizontal plane defined through longitudinal axis “X,” and terminates at a location offset below the horizontal plane defined through longitudinal axis “X.” Lower portion 455b of second proximal flag 454, on the other hand, extends from upper portion 455a at a location offset above the longitudinal axis “X” in a downward direction but terminates at a location offset above the horizontal plane defined through longitudinal axis “X” and, thus, does not cross the horizontal plane defined through longitudinal axis “X.” In other aspects, lower portion 455b of second proximal flag 454 extends to the horizontal plane defined through longitudinal axis “X.” As a result of this difference in extent of lower portions 453b, 455b of first and second proximal flags 452, 454, respectively, first proximal flag 452 defines a height greater than the height of second proximal flag 454.

Lower portion 453b of first proximal flag 452 includes a cam 422 protruding substantially perpendicularly in outward and/or inward directions from lower portion 453b of first proximal flag 452. Cam 422 is configured to operably couple jaw member 164 to second arm 420 of jaw actuation mechanism 400 and, thus, to the distal end portion of outer shaft 152. Cam 422 may be fixed to lower portion 453b of first proximal flag 452 such as, for example, within an aperture defined therethrough (via welding), attachment to inner and/or outer surfaces thereof (via monolithic formation or separate formation and subsequent attachment (e.g., via welding)), or in any other suitable manner. Alternatively, cam 422 may be rotatably mounted to lower portion 453b of first proximal flag 452. Cam 422 may include any suitable camming structure(s) such as, for example, a continuous cam pin, a split cam pin, separate cam pins, cam bosses, etc.

The pivotable mounting of jaw member 164 relative to inner shaft 153 via pivot 412 and the operable coupling of jaw member 164 to outer shaft 152 via cam 422 enables actuation of jaw member 164 in response to movement of outer shaft 152 about inner shaft 153. More specifically, sliding of outer shaft 152 about and relative to inner shaft 153 urges cam 422 relative to lower portion 453b of first proximal flag 452 to thereby pivot jaw member 164 about pivot 412 and relative to ultrasonic blade 162 between the open position and the closed position. Jaw actuation mechanism 400 is configured as a pull to close mechanism, wherein proximal movement of outer shaft 152 pivots jaw member 164 towards the closed position (and, thus, distal movement of outer shaft 152 pivots jaw member 164 towards the open position), although a push to close mechanism (wherein distal movement of outer shaft 152 pivots jaw member 164 towards the closed position and, thus, proximal movement of outer shaft 152 pivots jaw member 164 towards the open position) is also contemplated. Further, as noted above, it is contemplated that, in aspects, outer shaft 152 remain stationary while inner shaft 153 is movable to provide the above-detailed pivoting of jaw member 164 relative to ultrasonic blade 162.

Continuing with reference to FIGS. 4-6B, distal body 456 of structural frame 450 of jaw member 164 extends distally from proximal flags 452, 454 and is configured to at least partially conform to the curvature of ultrasonic blade 162 to enable clamping of tissue between jaw member 164 and ultrasonic blade 162 at any position along the length of the exposed portion of ultrasonic blade 162 (e.g., the portion of ultrasonic blade 162 that extends distally from jaw actuation mechanism 400). Distal body 456 retains jaw liner 460 thereon in any suitable manner such as, for example, forging, adhering, overmolding, mechanical capture, combinations thereof, etc.

With reference to FIGS. 6A and 6B, jaw liner 460 is positioned to oppose and align with ultrasonic blade 162 (and, in aspects, contact ultrasonic blade 162 in the closed position of jaw member 164) and is fabricated from a compliant material such as, for example, polytetrafluoroethylene (PTFE), silicone, or other suitable compliant material. The compliance of jaw liner 460 together with the alignment thereof relative to ultrasonic blade 162 enables the clamping of tissue between jaw liner 460 and ultrasonic blade 162 and also allows ultrasonic blade 162 to vibrate while in contact with jaw liner 460 (direct contact or indirect contact with tissue clamped therebetween) without damaging components of ultrasonic surgical instrument 100 (FIG. 1) and without compromising the hold on tissue clamped between jaw member 164 and ultrasonic blade 162.

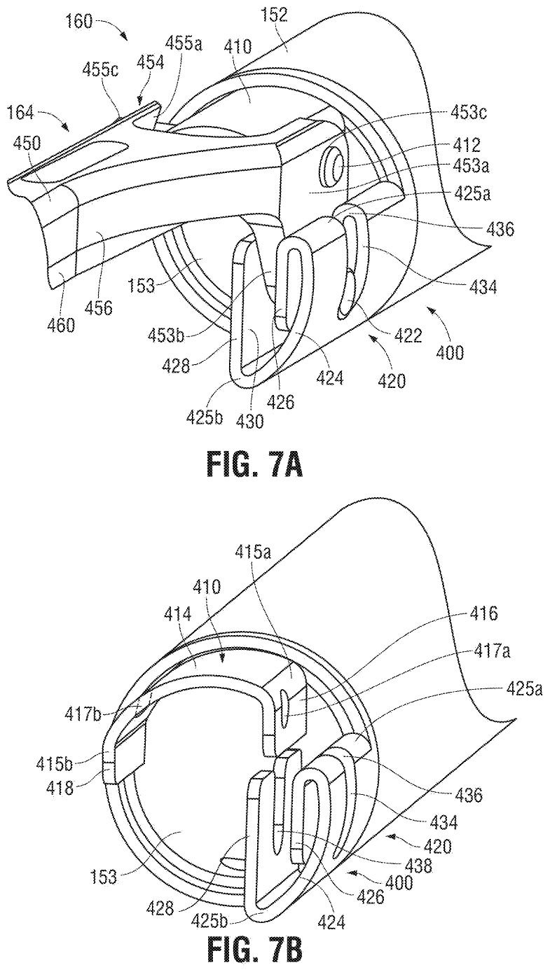

Referring to FIGS. 7A and 7B, in conjunction with FIGS. 4-6B, jaw actuation mechanism 400 is described. Jaw actuation mechanism 400, as noted above, includes first arm 410 extending from inner shaft 153 to pivotably mount jaw member 164 at the distal end portion of inner shaft 153 and second arm 420 extending from outer shaft 152 to operably couple jaw member 164 to the distal end portion of outer shaft 152.

First arm 410 may be formed from inner shaft 153 such that first arm 410 and inner shaft 153 are monolithically formed of the same material and of the same thickness. With respect to formation of first arm 410 from inner shaft 153, more specifically, while the body of inner shaft 153 is generally tubular, a distal end portion of the tubular inner shaft 153 may be selectively manipulated via cutting (e.g., laser cutting), material removal, and/or bending (e.g., stamping) to form the distal end portion of the tubular inner shaft 153 into first arm 410. However, other configurations are also contemplated such as, for example, wherein first arm 410 is separately formed and subsequently attached (e.g., via welding) to the distal end of inner shaft 153.

First arm 410 includes a curved body 414 that is attached to and extends distally from the tubular inner shaft 153. Cam body 414 is aligned with and defines a radius of curvature substantially equal to the radius of the tubular inner shaft 153 such that curved body conforms to the outer profile of inner tubular shaft 153. Curved body 414 is asymmetrically positioned relative to a vertical plane defined through longitudinal axis “X” such that the first and second side edges 415a, 415b, respectively, of curved body 414 are not aligned with one another. First arm 410 further includes first and second flags 416, 418, respectively, depending from the respective first and second side edges 415a, 415b of curved body 414. First and second flags 416, 418 define substantially planar configurations and are oriented substantially vertically. However, due at least in part to the above-detailed asymmetric configuration of curved body 414, first and second flags 416, 418 are offset from one another. More specifically, first flag 416 laterally opposes a portion of curved body 414 of first arm 410 while first arm 410 does not include any portion that laterally opposes second flag 418. That is, second flag 418 extends further towards a horizontal plane defined through longitudinal axis “X” as compared to first flag 416 or any other portion of first arm 410. With first flag 416 extending a lesser extent as compared to second flag 418 as detailed above, clearance is provided for second arm 420, as detailed below.

First flag 416 and curved body 414 define aligned pivot apertures 417a, 417b configured to align with one another and pivot apertures 453c, 455c of structural frame 450 of jaw member 164 for receipt of pivot 412 through pivot apertures 417a, 417b, 453c, 455c to pivotably mount jaw member 164 to first arm 410 of jaw actuation mechanism 400 with first and second proximal flags 452, 454 of structural frame 450 of jaw member 164 disposed outwardly of first and second flags 416, 418 of first arm 410, although other configurations are also contemplated (such as, for example, where first and second proximal flags 452, 454 of structural frame 450 of jaw member 164 are nested between first and second flags 416, 418 of first arm 410, or where first and second proximal flags 452, 454 of structural frame 450 of jaw member 164 define an offset configuration wherein one of first and second proximal flags 452, 454 of jaw member 164 is disposed inside the corresponding flag 416, 418 of first arm 410 and the other of first and second proximal flags 452, 454 of jaw member 164 is disposed outside the corresponding flag 416, 418 of first arm 410).

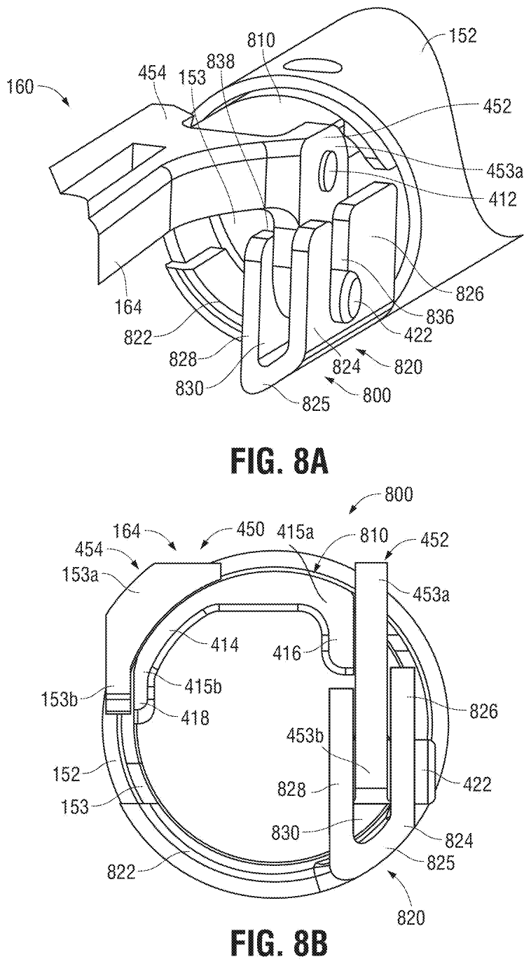

With jaw member 164 pivotably mounted to first arm 410 as detailed above, first flag 416 of first arm 410 is positioned adjacent to and in substantially parallel orientation relative to upper portion 453a of first proximal flag 452 of structural frame 450 of jaw member 164, second flag 418 of first arm 410 is positioned adjacent to and in substantially parallel orientation relative to lower portion 455b of second proximal flag 454 of structural frame 450 of jaw member 164, and upper portion 455a of second proximal flag 454 is positioned adjacent to and is substantially complementary to a portion of curved body 414. These adjacent and parallel or complementary structures function as guide features to maintain alignment of jaw member 164 and inhibit tilting or splay of jaw member 164 as jaw member 164 is actuated between the open and closed position via jaw actuation mechanism 400 (see also FIG. 8B). In particular, the substantially complementary curvatures of upper portion 455a of second proximal flag 454 and curved body 414 of first arm 410 function as guide structures on the second side of the vertical plane defined through longitudinal axis “X” to provide additional stability and support to counteract the effects of second arm 420 being disposed on and coupling to jaw member 164 on the first second side of the vertical plane defined through longitudinal axis “X” (see also FIG. 8B).

With continued reference to FIGS. 7A and 7B, in conjunction with FIGS. 4-6B, second arm 420 of jaw actuation mechanism 400 extends distally from outer shaft 152. Second arm 420 may be formed from outer shaft 152 such that second arm 420 and outer shaft 152 are monolithically formed of the same material and of the same thickness. With respect to formation of second arm 420 from outer shaft 152, more specifically, while the body of outer shaft 152 is generally tubular, a distal end portion of the tubular outer shaft 152 may be selectively manipulated via cutting (e.g., laser cutting), material removal, and/or bending (e.g., stamping) to form the distal end portion of the tubular outer shaft 152 into second arm 420. However, other configurations are also contemplated such as, for example, wherein second arm 420 is separately formed and subsequently attached (e.g., via welding) to the distal end of outer shaft 152.

Second arm 420 includes a curved body 424 and first and second uprights 426, 428. Curved body 424 is attached to and extends distally from the tubular outer shaft 152, defines a radius of curvature substantially equal to the radius of the tubular outer shaft 152, and includes upper and lower edges 425a, 425b, respectively. First and second uprights 426, 428 extend from respective upper and lower edges 425a, 425b of curved body 424. More specifically, second arm 420 is bent approximately 180 degrees at upper edge 425a of curved body 424 such that first upright 426 extends vertically in inwardly spaced-apart relation from curved body 424. First upright 426, in aspects, defines a substantially planar configuration and, as noted above, is oriented substantially vertically.

Second arm 420 is bent at lower edge 425b at an angle at least about 90 degrees but less than 180 degrees (owing to the curvature of curved body 424) such that second upright 428 extends vertically in inwardly spaced-apart and substantially parallel relation relative to first upright 426. Second upright 428, in aspects, defines a substantially planar configuration and, as noted above, is oriented substantially vertically. Other configurations are also contemplated.

As a result of the above-detailed configuration wherein first and second uprights 426, 428 extend in opposite directions in spaced-apart and substantially parallel relation relative to one another, a channel 430 is defined between first and second uprights 426, 428 for receipt of lower portion 453b of first proximal flag 452 of structural frame 450 of jaw member 164. The transverse width of channel 430 may substantially approximate the thickness of lower portion 453b of first proximal flag 452 of structural frame 450 of jaw member 164 to inhibit slop, thus helping to maintain alignment of jaw member 164 and inhibit tilting or splay of jaw member 164 as jaw member 164 is actuated between the open and closed position via jaw actuation mechanism 400. Additionally, first upright 426, as it is bent back along curved body 424, not only provides additional structural support to second arm 420 but may function as a spring finger to provided bias towards a reduced width of channel 430 (and/or to provide bias to inhibit expansion of the width of channel 430), thus additionally helping to maintain alignment of jaw member 164 and inhibit tilting or splay of jaw member 164.

At least first upright 426 and, in aspects, curved body 424 and/or second upright 428, define slots 436, 434, 438, respectively, therein. Slots 436, 434, 438 may be substantially vertical or, in other aspects, may be curved and/or angled, e.g., to modulate force applied to tissue via jaw member 164 and/or to guide parallel (or more parallel) closure of jaw member 164 relative to blade 162. In aspects where curved body 424 includes slot 434, a slot may be formed in second arm 420 prior to bending to form first upright 426 such that, upon bending, slots 436, 434 are formed sharing a common open upper end. Alternatively, slots 436, 434 may define separate (open or closed) configurations. Slot 438 of second upright 428, in aspects where provided, may define an open upper end (as shown) or may alternatively define a closed configuration. Slots 436, 434, 438 are configured to receive cam 422 when lower portion 453b of first proximal flag 452 is received within channel 430 to thereby operably couple jaw member 164 and second arm 420 (and thus, outer shaft 152) with one another.

With cam 422 received within slots 436, 434, 438, the walls (e.g., substantially vertical or otherwise configured walls) defining slots 436, 434, 438 urge cam 422 distally or proximally in response to movement of outer shaft 152 (wherein the direction of urging depends upon the direction of movement of outer shaft 152) to thereby pivot jaw member 164 about pivot 412 and relative to ultrasonic blade 162. While slots 436, 434, 438 substantially constrain cam 422 longitudinally, slots 436, 434, 438 enable substantially vertical (or, in aspects, angled or curved) sliding of cam 422 therethrough such that cam 422 can follow a substantially radiused path as it orbits about pivot 412 during the pivoting of jaw member 164 between the open and closed positions.

Turning to FIGS. 8A and 8B, another jaw actuation mechanism 800 configured for use with end effector assembly 160 of surgical instrument 100 of surgical system 10 (FIG. 1) is detailed, although jaw actuation mechanism 800 may similarly be utilized with surgical instrument 20 (FIG. 2), robotic surgical system 1000 (FIG. 3) and/or any other suitable surgical instrument or system. Further, jaw actuation mechanism 800 is similar to and may include any of the features of jaw actuation mechanism 400 (FIGS. 4-7B), except as explicitly contradicted hereinbelow. Accordingly, for purposes of brevity, only differences between jaw actuation mechanism 800 and jaw actuation mechanism 400 (FIGS. 4-7B) are described in detail below while similarities are summarily described or omitted entirely.

Jaw actuation mechanism 800 includes a first arm 810 extending from inner shaft 153 and a second arm 820 extending from outer shaft 152. First arm 810 may be identical to first arm 410 (FIGS. 4-7B) as detailed above and, thus, some of the features of first arm 810 are labeled with the same reference numerals detailed above with respect to first arm 410 (FIGS. 4-7B). Second arm 820 is separately formed from outer shaft 152 and attached to outer shaft 152 during manufacturing to allow second arm 820, in aspects, to define a wall thickness greater than a wall thickness of outer shaft 152, although other configurations including monolithic formation configuration are also contemplated.

Second arm 820 includes a partially annular lip 822 and a substantially U-shaped body 824. Annular lip 822 is configured to overlap a portion of the inner surface of outer shaft 152 and/or a distal face of outer shaft 152 to provide suitable surface area to fix second arm 820 relative to outer shaft 152, e.g., via welding, adhesion, mechanical engagement, or in any other suitable manner.

Substantially U-shaped body 824 of second arm 820 extends distally from annular lip 822 and defines a base 825 that is aligned with outer shaft 152 and has a radius of curvature that is substantially equal to the radius of outer shaft 152, although other configurations are also contemplated such as, for example, configurations wherein base 825 is substantially flat. First and second uprights 826, 828 extend from opposite ends of body 824 and define substantially planar configurations that are oriented substantially vertically and substantially parallel relative to one another to define a channel 830 therebetween. Channel 830 is configured for receipt of lower portion 453b of first proximal flag 452 of structural frame 450 of jaw member 164.

First and second uprights 826, 828 further include slots 836, 838 defined therein, which may be open or closed end slots. Slots 836, 838 are configured to receive cam 422 when lower portion 453b of first proximal flag 452 is received within channel 830 to thereby operably couple jaw member 164 and second arm 820 (and thus, outer shaft 152) with one another, similarly as detailed above with respect to jaw actuation mechanism 400 (FIGS. 4-7B).

Aspects of this disclosure may be further described by reference to the following numbered paragraphs:

1. An ultrasonic surgical instrument (100), comprising: an ultrasonic blade (162) including a top side (436), a bottom side (438), a first lateral side (434), and a second lateral side (432); a jaw member (164) movable between an open position and a closed position for clamping tissue against the top side (436) of the ultrasonic blade (162), the jaw member (164) including a first proximal flag (452) disposed on the second lateral side (432) of the ultrasonic blade (162) and a second proximal flag (454) disposed on the first lateral side (434) of the ultrasonic blade (162), wherein a portion of the first proximal flag (452) extends beyond the second proximal flag (454); and a jaw actuation mechanism (400, 800) including: a first arm (410, 810) pivotably connected to the first proximal flag (452) and the second proximal flag (454) on the top side (436) of the ultrasonic blade (162); and a second arm (420, 820) operably coupled to the portion of the first proximal flag (452) on the second lateral side (432) of the ultrasonic blade (162), wherein movement of at least one of the first arm (410, 810) or the second arm (420, 820) relative to another of the first arm (410, 810) or the second arm (420, 820) moves the jaw member (164) between the open position and the closed position.

2. The ultrasonic surgical instrument (100) according to paragraph 1, wherein the second arm (420, 820) includes a first upright and a second upright defining a channel (430, 830) therebetween, and wherein the portion of the first proximal flag (452) is received within the channel (430, 830).

3. The ultrasonic surgical instrument (100) according to paragraph 2, wherein at least one of the first upright or the second upright defines a slot (436, 434, 438, 836, 838) and wherein the portion of the first proximal flag (452) includes a cam (422) received within the slot (436, 434, 438, 836, 838).

4. The ultrasonic surgical instrument (100) according to paragraph 2 or 3, wherein the first upright defines a spring finger (426) configured to maintain the portion of the first proximal flag (452) in alignment within the channel (430).

5. The ultrasonic surgical instrument (100) according to any preceding paragraph, further comprising a first shaft (154) and a second shaft (152), the first arm (410, 810) fixed relative to and extending distally from the first shaft (154) and the second arm (420, 820) fixed relative to and extending distally from the second shaft (152), wherein one of the first shaft (154) or the second shaft (152) is movable relative to another of the first shaft (154) or the second shaft (152) to thereby move the at least one of the first arm (410, 810) or the second arm (420, 820) relative to the other of the first arm (410, 810) or the second arm (420, 820).

6. The ultrasonic surgical instrument (100) according to paragraph 5, wherein: the first arm (410, 810) includes a portion that conforms to a portion of an outer periphery of the first shaft (154); or the second arm (420, 820) includes a portion that conforms to a portion of an outer periphery of the second shaft (152).

7. The ultrasonic surgical instrument (100) according to paragraph 5, wherein: the first arm (410, 810) includes a portion that conforms to a portion of an outer periphery of the first shaft (154); and the second arm (420, 820) includes a portion that conforms to a portion of an outer periphery of the second shaft (152).

8. The ultrasonic surgical instrument (100) according to any preceding paragraph, wherein the first proximal flag (452) defines a substantially planar configuration and wherein the second proximal flag (454) includes a curved portion.

9. The ultrasonic surgical instrument (100) according to paragraph 8, wherein the first arm (410, 810) includes a curved portion and wherein the curved portion of the second proximal flag (454) is complementary to the curved portion of the first arm (410, 810).

10. The ultrasonic surgical instrument (100) according to paragraph 9, wherein the curved portions of the first arm (410, 810) and the second proximal flag (454) cooperate to guide the jaw member (164) during movement of the jaw member (164) between the open position and the closed position.

11. The ultrasonic surgical instrument (100) according to any one of paragraphs 8-10, wherein the second proximal flag (454) further includes a substantially planar portion extending from the curved portion and wherein the first arm (410, 810) includes a substantially planar portion extending from the curved portion.

12. The ultrasonic surgical instrument (100) according to paragraph 11, wherein the substantially planar portions of the first arm (410, 810) and the second proximal flag (454) cooperate to guide the jaw member (164) during movement of the jaw member (164) between the open position and the closed position.

13. The ultrasonic surgical instrument (100) according to any one of paragraphs 7-12, wherein a pivot (412) pivotably connects the first arm (410, 810) to the first proximal flag (452) and the curved portion of the second proximal flag (454).

14. The ultrasonic surgical instrument (100) according to any preceding paragraph, wherein the ultrasonic blade (162) is curved such that the first lateral side (434) is convex and the second lateral side (432) is concave.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. An ultrasonic surgical instrument, comprising:
a curved ultrasonic blade including a top side, a bottom side, a convex side, and a concave side;
a jaw member movable between an open position and a closed position for clamping tissue against the top side of the curved ultrasonic blade, the jaw member including a first proximal flag disposed on the concave side of the curved ultrasonic blade and a second proximal flag disposed on the convex side of the curved ultrasonic blade, the first proximal flag defining a height greater than a height of the second proximal flag such that a portion of the first proximal flag extends beyond the second proximal flag; and
a jaw actuation mechanism including:
a first arm pivotably connected to the first proximal flag and the second proximal flag on the top side of the curved ultrasonic blade; and
a second arm operably coupled to the portion of the first proximal flag on the concave side of the curved ultrasonic blade,
wherein movement of at least one of the first arm or the second arm relative to another of the first arm or the second arm moves the jaw member between the open position and the closed position.

2. The ultrasonic surgical instrument according to claim 1, wherein the second arm includes a first upright and a second upright defining a channel therebetween, and wherein the portion of the first proximal flag is received within the channel.

3. The ultrasonic surgical instrument according to claim 2, wherein at least one of the first upright or the second upright defines a slot and wherein the portion of the first proximal flag includes a cam received within the slot.

4. The ultrasonic surgical instrument according to claim 2, wherein the first upright defines a spring finger configured to maintain the portion of the first proximal flag in alignment within the channel.

5. The ultrasonic surgical instrument according to claim 1, further comprising a first shaft and a second shaft, the first arm fixed relative to and extending distally from the first shaft and the second arm fixed relative to and extending distally from the second shaft, wherein one of the first shaft or the second shaft is movable relative to another of the first shaft or the second shaft to thereby move the at least one of the first arm or the second arm relative to the other of the first arm or the second arm.

6. The ultrasonic surgical instrument according to claim 5, wherein at least one of:
the first arm includes a portion that conforms to a portion of an outer periphery of the first shaft; or
the second arm includes a portion that conforms to a portion of an outer periphery of the second shaft.

7. The ultrasonic surgical instrument according to claim 1, wherein the first proximal flag defines a substantially planar configuration and wherein the second proximal flag includes a curved portion.

8. The ultrasonic surgical instrument according to claim 7, wherein the first arm includes a curved portion and wherein the curved portion of the second proximal flag is complementary to the curved portion of the first arm such that the curved portions of the first arm and the second proximal flag cooperate to guide the jaw member during movement of the jaw member between the open position and the closed position.

9. The ultrasonic surgical instrument according to claim 8, wherein the second proximal flag further includes a substantially planar portion extending from the curved portion, wherein the first arm includes a substantially planar portion extending from the curved portion, and wherein the substantially planar portions of the first arm and the second proximal flag cooperate to guide the jaw member during movement of the jaw member between the open position and the closed position.

10. The ultrasonic surgical instrument according to claim 7, wherein a pivot pivotably connects the first arm to the first proximal flag and the curved portion of the second proximal flag.

11. An ultrasonic surgical instrument, comprising:
a shaft assembly including a first shaft and a second shaft, at least one of the first shaft or the second shaft movable relative to another of the first shaft or the second shaft;
an ultrasonic blade extending distally from the shaft assembly and including a top side, a bottom side, a first lateral side, and a second lateral side; and
a jaw member extending distally from the shaft assembly, the jaw member pivotably mounted relative to the second shaft about a pivot and movable about the pivot between an open position and a closed position for clamping tissue against the top side of the ultrasonic blade,
wherein the jaw member is operably coupled to first shaft only on the first lateral side of the ultrasonic blade, the jaw member operably coupled to first shaft such that the movement of the at least one of the first shaft or the second shaft relative to the other of the first shaft or the second shaft moves the jaw member about the pivot between the open position and the closed position.

12. The ultrasonic surgical instrument according to claim 11, wherein an actuation arm operably couples the jaw member with the first shaft only on the first lateral side of the ultrasonic blade, the actuation arm including a first upright and a second upright defining a channel therebetween, and the jaw member including a flag received within the channel.

13. The ultrasonic surgical instrument according to claim 12, wherein at least one of the first upright or the second upright defines a slot and wherein the flag includes a cam received within the slot.

14. The ultrasonic surgical instrument according to claim 12, wherein the first upright defines a spring finger configured to maintain the flag in alignment within the channel.

15. The ultrasonic surgical instrument according to claim 12, wherein the actuation arm is fixed relative to and extends distally from the first shaft.

16. The ultrasonic surgical instrument according to claim 12, wherein a portion of the actuation arm defines an extension of an outer periphery of a portion of the first shaft.

17. The ultrasonic surgical instrument according to claim 11, wherein a pivot arm pivotably couples the jaw member with the second shaft, the pivot arm disposed on the top side of the ultrasonic blade.

18. The ultrasonic surgical instrument according to claim 17, wherein the pivot arm and the jaw member define complementary at least partially curved guide surfaces only on the second lateral side of the ultrasonic blade.

19. The ultrasonic surgical instrument according to claim 17, wherein a portion of the pivot arm defines an extension of an outer periphery of a portion of the second shaft.

20. The ultrasonic surgical instrument according to claim 11, wherein the ultrasonic blade is curved such that the first lateral side is concave and the second lateral side is convex.
